(19) United States
(12) Patent Application Publication
(10) Pub. No.: US 2023/0338533 A1
(43) Pub. Date: Oct. 26, 2023

(54) Hypoimmunogenic Cells and Uses Thereof in Immune Responses

(71) Applicant: RXCELL INC., Novato, CA (US)

(72) Inventors: Xianmin ZENG, Novato, CA (US); Mahendra RAO, Timonium, MD (US)

(21) Appl. No.: 17/923,070

(22) PCT Filed: May 13, 2021
(86) PCT No.: PCT/US2021/032229

§ 371 Date: Nov. 3, 2022

Related U.S. Application Data
(60) Provisional application No. 63/025,351, filed on May 15, 2020.

(51) Int. Cl. A61K 39/00; A61P 37/04; A61K 39/215

(52) U.S. Cl. CPC A61K 39/4633; A61P 37/04; A61K 39/461; A61K 39/215

(57) ABSTRACT

Hypoimmunogenic cell lines useful in enhancing, invoking and/or stimulating an immune response and methods for their use and production are provided.

This patent application claims the benefit of priority from U.S. Application Ser. No. 63/025,351, filed May 15, 2020, teachings of which are incorporated herein by reference in their entirety.

### FIELD

The present invention relates to hypoimmunogenic cell lines, compositions comprising the hypoimmunogenic cells lines, and methods for their use as vaccines and in enhancing, invoking and/or stimulating an immune response.

### BACKGROUND

The immune system plays an important role in maintaining the integrity of the organism. It recognizes self from non-self and mounts a defense against bacterial, viral and other microorganisms. The immune system is comprised of cells in the circulation as well as resident immune cells present in most tissues and organs.

The immune system recognizes foreign antigens via two major arms of the immune system (Chaplin et al. J Allergy Clin Immunol. 2010 February; 125(2 Suppl 2):S3-23). The mechanisms permitting recognition of microbial, toxic, or allergenic structures can be broken down into two general categories referred to as innate and adaptive immune pathways.

Innate immune pathways are hard-wired responses that are encoded by genes in the host's germ line and that recognize molecular patterns shared both by many microbes and toxins that are not present in the mammalian host. The innate response also includes soluble proteins and bioactive small molecules that are either constitutively present in biological fluids (such as complement proteins, defensins, and ficolins (Hiemstra, P. S. Exp Lung Res. 2007; 33:537-542; Holmskov et al. Annu Rev Immunol. 2003; 21:547-578) or that are released from cells as they are activated (including cytokines that regulate the function of other cells, chemokines that attract inflammatory leukocytes, lipid mediators of inflammation, reactive free radical species, and bioactive amines and enzymes that also contribute to tissue inflammation). The first set of responses constitutes the innate immune response because the recognition molecules used by the innate system are expressed broadly on a large number of cells. This system is poised to act rapidly after an invading pathogen or toxin is encountered and thus constitutes the initial host response.

The second set of immune responses constitutes the adaptive immune system, also referred as the acquired immune system. Unlike the innate immune system, the acquired immune system is highly specific to a particular pathogen. Adaptive immune pathway responses are encoded by gene elements that somatically rearrange to assemble antigen-binding molecules with exquisite specificity for individual unique foreign structures. The human leukocyte antigen (HLA) system or complex is a gene complex encoding the major histocompatibility complex (MHC) proteins in humans. These cell-surface proteins are responsible for the regulation of the immune system in humans.

The innate and adaptive immune systems are often described as contrasting, separate arms of the host response; however, they usually act together, with the innate response representing the first line of host defense, and with the adaptive response becoming prominent after several days, as antigen-specific T and B cells have undergone clonal expansion. Components of the innate system contribute to activation of the antigen-specific cells. Additionally, the antigen-specific cells amplify their responses by recruiting innate effector mechanisms to bring about the complete control of invading microbes. Thus, while the innate and adaptive immune responses are fundamentally different in their mechanisms of action, synergy between them is essential for an intact, fully effective immune response.

Other important components of the immune system are derived from the hematopoietic stem cells. Multipotent hematopoietic stem cells differentiate in bone marrow into common lymphoid or common myeloid progenitor cells. Lymphoid stem cells give rise to B cell, T cell, and NK cell lineages. Myeloid stem cells give rise to a second level of lineage specific colony form unit (CFU) cells that go on to produce neutrophils, monocytes, eosinophils, basophils, mast cells, megakaryocytes, and erythrocytes. Monocytes differentiate further into macrophages in peripheral tissue compartments. Dendritic cells (DC) appear to develop primarily from a DC precursor that is distinguished by its expression of the Flt3 receptor. This precursor can derive from either lymphoid or myeloid stem cells and gives rise to both classical DC and plasmacytoid DC. Classical DC can also derive from differentiation of monocytoid precursor cells.

T cells are also an important arm of the immune system. T cells have evolved an elegant mechanism that recognizes foreign antigens together with self-antigens as a molecular complex. This requirement that T cells recognize both self-structures and foreign antigens makes the need for these cells to maintain self-tolerance particularly important.

A major role of the T cell arm of the immune response is to identify and destroy infected cells. T cells can also recognize peptide fragments of antigens that have been taken up by antigen presenting cells (APC) through the process of phagocytosis or pinocytosis. The way the immune system has evolved to permit T cells to recognize infected host cells is to require that the T cell recognize both a self-component and a microbial structure. The elegant solution to the problem of recognizing both a self-structure and a microbial determinant is the family of MHC molecules. MHC molecules (also called the HLA antigens) are cell surface glycoproteins that bind peptide fragments of proteins that either have been synthesized within the cell (class I MHC molecules) or that have been ingested by the cell and proteolytically processed (class II MHC molecules).

There are three major HLA class I molecules, designated HLA-A, HLA-B, and HLA-C, each encoded by a distinct gene. The class I HLA molecules are cell surface heterodimers consisting of a polymorphic transmembrane 44-kd α-chain (also designated the class I heavy chain) associated with the 12-kd non-polymorphic β₂-microglobulin (β₂m) protein. The α-chain determines whether the class I molecule is an HLA-A, HLA-B, or HLA-C molecule. The HLA-A, HLA-B, and HLA-C α-chain genes are encoded within the MHC on chromosome 6, and the β₂-microglobulin gene is encoded on chromosome 15. The TCR contacts both the antigenic peptide and the flanking α-helices. The TCR has no measurable affinity for the antigenic peptide alone, and very low affinity for MHC molecules containing other peptides. It has been recognized that T cells only recognize their specific antigen when it was presented in association with a specific self-MHC molecule (Zinkernagel, R. M. and Doherty, P. C. (1977) Major Transplantation Antigens, Viruses, and Specificity of Surveillance T Cells. In: Stutman O. (eds) Contemporary Topics in Immunobiology. Contemporary Topics in Immunobiology, vol 7. Springer, Boston, MA).

Like the class I molecules, class II HLA molecules consist of two polypeptide chains, but in this case, both are MHC-encoded transmembrane proteins and are designated a and β. There are three major class II proteins designated HLA-DR, HLA-DQ, and HLA-DP. Molecules encoded in this region were initially defined serologically and using cellular immune assays, and consequently their nomenclature does not always reflect the underlying genes encoding the molecules. This is particularly true for HLA-DR, where the genes in the HLA-DR sub-region encode 1 minimally polymorphic (1 common and 2 very rare alleles) a chain (designated DRA) and 2 polymorphic β chains (designated DRB1 and DRB3).

Endogenous proteins are digested by the immunoproteasome to small peptide fragments optimal for loading into Class I molecules. Peptides are transferred from the immunoproteasome to the endoplasmic reticulum via the TAP transporter. With the help of tapscin, calreticuin and the chaperon Erp57, they are loaded into a class I heavy chain that associates with a β₂m subunit via facilitation by chaperon protein calnexin prior to transport to the cell surface wherein it can be recognized by CD8+ T cells.

Recently it has also become clear that there is a class of T cells that recognizes antigens presented by molecules that are not classical HLA class I or class II antigens. One of these classes of T cells uses an antigen receptor composed of α and β chains and recognizes lipid antigens that are presented bound to CD1 molecules. CD1 molecules are structurally related to class I HLA molecules, being transmembrane proteins with 3 extracellular domains and associating with β₂-microglobulin. There are 5 human CD1 isoforms designated CD1α-CD1e, encoded by linked genes that are not associated with the MHC.

Additional innate immune effectors critical for effective host defense include phagocytic cells including neutrophils, macrophages, and monocytes and natural killer (NK) cells.

Modern gene editing technologies to knock out expression of all Class I and Class II genes have been used in an attempt to generate universal cells to prevent immune rejection (Lanza, Russell and Nagy 2019). Modifications of this strategy with largely the same goals have been used by a variety of other investigators including using other variants of HLA-G, using HLA-E or knocking out each HLA-class individually or assuming Class II knock out will not be necessary. In general, while these strategies substantially inhibited adaptive immunity there was only a modest reduction or an increase in the innate immune response leading to chronic rejection.

Vaccines are produced in a variety of ways with their efficacy primarily based upon their ability to induce durable production of protective pathogen-specific antibodies by plasma and memory B cells. Coordination of the innate and adaptive immune systems is paramount to the development of protective humoral and cellular immunity following vaccination.

Traditionally, vaccines have consisted of inactivated organisms or protein products.

Vaccine generation is more effective when live cells are used. Cell lines delivering processed antigen elicit a better immune response and it has been suggested that activating the NK cell pathway to initiate apoptosis may elicit a better immune response.

A subset of vaccines for cancer therapy have been produced by using cells that express similar antigens. Wu and colleagues made the observation that pluripotent stem cells express antigens that are related to cancers and thus immunization with the cells should elicit an anti-tumor response. Initial experiments were performed with embryonic stem cells and subsequently with induced pluripotent stem cells (iPSC).

Using dead or dying cells (apoptotic cells) which naturally provide peptide antigens has been shown to elicit a stronger immune response than using lysed cells or protein antigens alone. Thus, it has been suggested that presenting antigen via live cells that are undergoing apoptosis may be an effective vaccination process.

A major problem with these approaches, however, is that cells provide a multitude of antigens that may or may not include epitopes against the antigen of interest. Use of autologous cells, however, reduces the immune response and may induce autoantibodies if other stimulatory signals are used.

Accordingly, there is a need for improved vaccines and compositions and methods for enhancing an immune response.

### SUMMARY

In this disclosure, hypoimmunogenic cells are used to enhance, invoke and/or stimulate an immune response. These hypoimmunogenic cells are protected from the adaptive immune response initially, thus surviving for a longer period in vivo and therefore presenting any foreign molecules expressed by the cells for a prolonged time period. However, the cells are ultimately recognized as foreign by the innate immune response and will be attacked by NK cells, thereby undergoing apoptosis and allowing for antigen presentation of the expressed foreign molecules to the adaptive immune system resulting in the generation of antibodies against any foreign antigen. Hypoimmunogenic cells of this disclosure can also be used as an adjuvant to delivery of protein or peptide antigens or other antigens to enhance the inflammatory response.

Accordingly, an aspect of the present invention relates to a cell modified to be hypoimmunogenic and to express one or more foreign molecules such as, but not limited to proteins, carbohydrates, nucleotides, cytokines, chemokines, antibodies and lectins. In one nonlimiting embodiment, the cell is modified to express a foreign molecule which is antigenic. In one nonlimiting embodiment, the cell is modified to be hypoimmunogenic by eliminating or lowering expression of HLA Class I epitopes and Class II epitopes.

Another aspect of the present invention relates to a vaccine against a foreign antigen comprising a cell modified to be hypoimmunogenic and to express the foreign antigen and a pharmaceutically acceptable carrier. In one nonlimiting embodiment, the cell is modified to be hypoimmunogenic by eliminating or lowering expression of Class I epitopes and Class II epitopes.

Another aspect of the present invention relates to methods for enhancing, invoking and/or stimulating an immune response against a foreign antigen in a mammal by administering to the mammal a cell modified to be hypoimmunogenic and to express a foreign molecule such as, but not limited to proteins, carbohydrates, nucleotides, cytokines, chemokines, antibodies and lectins. In one nonlimiting embodiment, the cell is modified to express the foreign antigen. In one nonlimiting embodiment, the cell is modified to be hypoimmunogenic by eliminating or lowering expression of Class I epitopes and Class II epitopes.

Another aspect of the present invention relates to a method for protecting a mammal from a disease or infection relating to a foreign antigen. The method comprises administering to the mammal a vaccine against the foreign antigen wherein the vaccine comprises a cell modified to be hypoimmunogenic and to express a foreign molecule such as, but not limited to proteins, carbohydrates, nucleotides, cytokines, chemokines, antibodies and lectins. In one nonlimiting embodiment, the cell is modified to express the foreign antigen. Vaccines administered may further comprise a pharmaceutically acceptable carrier. In one nonlimiting embodiment, the cell is modified to be hypoimmunogenic by eliminating or lowering expression of Class I epitopes and Class II epitopes.

Another aspect of the present invention relates to a method for producing a vaccine against a foreign antigen. The method comprises modifying a cell to be hypoimmunogenic and to express a foreign molecule such as, but not limited to proteins, carbohydrates, nucleotides, cytokines, chemokines, antibodies and lectins. In one nonlimiting embodiment, the cell is modified to express the foreign antigen. In one nonlimiting embodiment, the cell is modified to be hypoimmunogenic by eliminating or lowering expression of Class I epitopes and Class II epitopes.

Another aspect of the present invention relates to a vaccine comprising a cell modified to be hypoimmunogenic and an antigenic peptide or protein or nucleotide. In one nonlimiting embodiment, the cell is modified to be hypoimmunogenic by eliminating or lowering expression of Class I epitopes and Class II epitopes.

Yet another aspect of the present invention relates to a method for enhancing an immune response to an antigenic peptide or protein which comprises administering the antigenic peptide or protein with cells modified to be hypoimmunogenic. In one nonlimiting embodiment, the cell is modified to be hypoimmunogenic by eliminating or lowering expression of Class I epitopes and Class II epitopes.

### BRIEF DESCRIPTION OF THE FIGURES

FIG. 1 provides a nonlimiting example of a master design of generating HLA-KO cells expressing one or more foreign antigens for vaccines.

FIG. 2 provides a nonlimiting example of a method for rapid production of a population of cells expressing multiple antigens for antibody responses using a floxed construct inserted in a safe harbor to utilize cassette exchange to replace one antigen with another thereby producing multiple cell lines which express multiple epitopes. In this nonlimiting example, foreign gene (antigen) expression is illustrated by green fluorescent protein (GFP) in the first step which can be replaced by cassette exchange and the process can be repeated in different types of cells.

### DETAILED DESCRIPTION

Disclosed herein are cell lines modified to be hypoimmunogenic and to enhance, invoke and/or stimulate an immune response. Such cells are protected from the adaptive immune response initially, thus allowing for survival for a longer period in vivo and a prolonged time period for presentation of foreign molecules expressed by the cells. Further, as the cells will eventually be recognized as foreign by the innate immune response and will be attacked by NK cells, they will undergo apoptosis sending inflammatory signals to the professional antigen presenting cells (APC) which will pick up the foreign molecules along with other possible antigens such as would happen in an immune response to a viral or parasitic infection. This interaction between killing of cells via the innate pathway and the presentation of antigens by professional APC is considered critical to generation of antibodies to pathogens. It is expected that these hypoimmunogenic cells will be useful as vaccines generating sufficient levels of antibodies against foreign antigenic proteins, carbohydrates or nucleotides expressed by the cells thereby protecting an individual against a disease or infection relating to the foreign antigenic protein. Further, initial protection of such cells modified to express foreign molecules such as, but not limited to, cytokines, chemokines, antibodies and lectins, makes them useful in enhancing, invoking and/or stimulating an immune response.

By the term “hypoimmunogenic cell” as used herein it is meant to encompass any cell modified to be protected initially from the adaptive immune response, thus allowing for survival for a longer period in vivo and a prolonged time period for foreign antigen presentation but which is eventually recognized as foreign by the innate immune response and attacked, thereby undergoing so that antibodies are generated against antigens expressed by or administered with the cells.

In one nonlimiting embodiment of the present invention, the hypoimmunogenic cell line is an iPSC or an immortalized line. In one nonlimiting embodiment, the hypoimmunogenic cells are HLA Class I and Class II null cells. A nonlimiting example is HLA Class I and II KO (HLA-null) mesenchymal stem cells (MSC). In this nonlimiting example, most of the antigens presented are similar to other endogenous human proteins so they will not elicit an immune response and no antibodies should be raised against them. However, the foreign highly immunogenic epitopes specifically expressed by HLA-null MSC will be targeted by the immune system for an enhanced and focused immune response with the development of both memory cells and antibodies to the target epitope. The co-opting of the normal innate immune pathway leading to antibody development against infectious agents of HLA-null MSC of the present invention is different from the mechanism of action of unmodified MSC where activation and overexpression of MHC class I and II receptors that is provoked with proinflammatory cytokines to enhance antigen delivery and cytokine activation of the immune system. See Tomchuck et al. Frontiers in Cellular and Infection Microbiology 2012 2(140):1-8.

In one nonlimiting embodiment, the hypoimmunogenic cells are modified via methods such as, but not limited to, antisense or miRNA to lower the cell's HLA expression.

In one nonlimiting embodiment, Class I and/or Class II knockout cell lines of the present invention, such as, but not limited to, B2M and CTIIA cell lines, are created which express a foreign antigen. Foreign antigen expression can be created using either a safe harbor locus integration (targeted integration) or via random integration for expressing one or more foreign antigens.

In one nonlimiting embodiment of the present invention, knocking out the Class I locus is achieved by knocking out B2M gene which is a component of the Class I molecules. In another nonlimiting embodiment, knocking out the Class I locus is achieved by knocking out genes (HLA-A, HLA-B and HLA-C) individually. In another nonlimiting embodiment, inhibiting expression of Class I antigens is achieved by knocking out processing enzymes such as, but not limited to, tapasin, which prevent surface expression of the Class I antigens.

In one nonlimiting embodiment, knocking out the Class II locus is achieved by knocking out a master transcription factor such as the RFANX or CIITA gene that is required for Class II gene expression.

In some nonlimiting embodiments, the hypoimmunogenic cells are further modified to express a foreign molecule such as, but not limited to, proteins, carbohydrates, nucleotides, cytokines, chemokines, antibodies and lectins. In one nonlimiting embodiment, the cells are modified to express a foreign antigenic protein. Nonlimiting examples of foreign antigens expressed by the hypoimmunogenic cells include viral antigens and other pathogens and antigens against tumor epitopes.

Nonlimiting examples of other molecules expressed by the cells include carbohydrates, nucleotides, cytokines, chemokines, antibodies and lectins. In one nonlimiting embodiment, the hypoimmunogenic cells are modified to express a monoclonal antibody. In one nonlimiting embodiment, the hypoimmunogenic cells are modified to express a therapeutic monoclonal antibody.

As carbohydrates, lipids and mixed structures are oftentimes carried on a protein scaffold to generate antigens to which many antibodies are targeted, expressing these foreign molecules in mammalian cells as in the present invention is advantageous as the carbohydrates, lipids and mixed structures are maintained as compared to other species.

Most of the antigens presented upon apoptosis of these modified cells are similar to other endogenous human proteins so they will not elicit an immune response and no antibodies should be raised against them. However, any foreign highly immunogenic epitopes specifically expressed by the engineered cells of the present invention will be targeted by the immune system thus producing an enhanced and focused immune response with the development of both memory cells and antibodies to the target epitope.

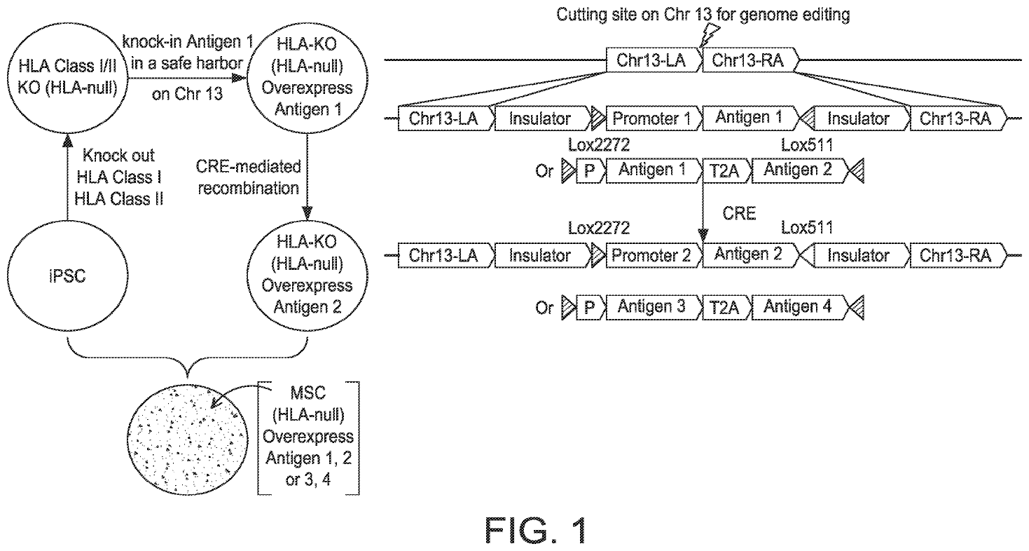

A nonlimiting example of a master design of generating HLA-KO cells expressing one or more foreign antigens for vaccines is set forth in FIG. 1. As shown therein, the hypoimmunogenic cells can be further modified to express one or more foreign antigens in a safe harbor site such as chromosome 13 and/or AAVS1 site on chromosome 19 using, from example T2A or IRES. Multiple foreign antigens can be expressed to increase antibody generation and pooled populations of cells can be used to generate antibodies to multiple epitopes. Further, use of CRE recombination allows for rapid generation of these cells for vaccine formulation.

Modified cells of the present invention can be administered to a mammal to induce an immune response against a foreign antigen expressed by the cell. In addition, the cells can be administered as a vaccine further comprising a pharmaceutically acceptable ingredient to a mammal to protect the mammal from a disease or infection relating to the foreign antigen.

Any mode of administration routinely used for vaccines can be used. Examples include, but are not limited to, subcutaneous, intramuscular or intravenous injection as well as oral, sublingual or nasal administration.

By “mammal”, as used herein, it is meant to include, but is not limited to, humans, primates, dogs, cats, rodents, horses, pigs, sheep, lagomorphs and cows.

Vaccine formulations of the present invention may be further enhanced by using additional activation agents such as foreign viral DNA or Poly I:C (Polyinosinic:polycytidylic acid) by simply expressing them in cells prior to administration or by delivering them in the vaccine formulation.

Further, while RNA and DNA vaccines suffer from the issue of delivery of sufficient material to cells for appropriate and persistent expression, such vaccines can be delivered ex-vivo to the HLA null cells of the present invention to enhance delivery by a log order and to enhance the antibody production process.

Hypoimmunogenic cells of the present invention can also be used to deliver molecules such as, but not limited to, cytokines, chemokines, antibodies and lectins expressed by the cells to enhance, invoke and/or stimulate an immune an immune response and as adjuvants with delivery of antigenic proteins or peptides antigens to enhance the inflammatory response. In one nonlimiting embodiment, the hypoimmunogenic cells are used to deliver a monoclonal antibody, more preferably a therapeutic monoclonal antibody expressed by the cell.

Various methods for producing these hypoimmunogenic cell lines can be used. Nonlimiting examples include safe harbor insertion and random integration by lentivirus and Piggy-Bac and sleeping beauty technologies.

The present invention also provides nucleic acid constructs for insertion or integration into the hypoimmunogenic cell line comprising a gene encoding the foreign antigen. Expression of the gene product can be driven by an endogenous promoter when the insertion is in frame with an endogenous gene, or by an exogenous promoter such as CAG or EF-alpha or any other well-known exogenously supplied promoter with the limitation that this be expressed in the cell product to which it is being inserted or integrated. The construct may be inserted into a known safe harbor locus such as the AAVS1 site or the Chr13 site. In accordance with the present invention, the nucleic acid construct is inserted or integrated into a cell line in with Class I epitopes and Class II epitopes have been knocked out.

An hypoimmunogenic cell line produced in accordance with the present invention with Class I and II double knock-out, and expressing a foreign antigen was produced.

Gene RNA guide information used to produce this cell line is provided in Tables 1 and 2.

TABLE 1
Gene and RNA guide information for B2M gene

| Gene Name | B2M |
|---|---|
| Transcript ID | ENST00000303088.8 |
| Guide RNA 1 Sequence | GGCCGAGAUGUCUCGCUCCG |
| | (SEQ ID NO: 1) |
| Guide RNA 2 Sequence | ACUCACGCUGGAUAGCCUCC |
| | (SEQ ID NO: 2) |
| Guide RNA 3 Sequence | CGGAGCGAGAGAGCACAGCG |
| | (SEQ ID NO: 3) |

PCR & Sequencing Primers:

| | |
|---|---|
| FOR primer (5′-3′) | ACAGCAAACTCACCCAGTCTAG |
| | (SEQ ID NO: 4) |
| REV Primer (5′-3′) | CCAGTCTAAGGGAAGCAGAGC |
| | (SEQ ID NO: 5) |
| GC Enhancer Used | N/A |
| Sequencing Primer Used | AAACTCACCCAGTCTAGTGC |
| | (SEQ ID NO: 6) |

TABLE 2
Gene and RNA guide information for CIITA gene

| Gene Name | CIITA |
|---|---|
| Transcript ID | ENST00000324288.12 |
| Guide RNA 1 Sequence | CACAGCUGAGCCCCCCACUG |
| | (SEQ ID NO: 7) |
| Guide RNA 2 Sequence | GGCUCCUGGUUGAACAGCGC |
| | (SEQ ID NO: 8) |
| Guide RNA 3 Sequence | CCCCUAACAUACUGGGAAUC |
| | (SEQ ID NO: 9) |

PCR & Sequencing Primers:

| | |
|---|---|
| FOR primer (5′-3′) | TGAGAGCTTGGGGTCCCTTA |
| | (SEQ ID NO: 10) |
| REV Primer (5′-3′) | CTGAGGCATGTTCTCTGCCA |
| | (SEQ ID NO: 11) |
| GC Enhancer Used | N/A |
| Sequencing Primer Used | GGTAGGGGCTTGGAGCTAAC |
| | (SEQ ID NO: 12) |

Double KO of the B2M and CIITA genes in both alleles was confirmed by DNA sequencing. Assessment of the morphology of the HLA-KO iPSC culture showed pluripotency of the HLA-KO iPSC culture, a normal karyotype and validation of short-tandem repeat (STR) of the HLA-KO iPSC line

Additional nonlimiting examples of cell lines which can be created in accordance with the present invention include RCL-BC-2, an HLA Class I (B2M) and Class II (CIITA) double knock-out iPSC line produced using NCL2 as the parental line and RCL-BC-2-GFP, an iPSC line generated by knocking-out both HLA Class I (B2M) and Class II (CIITA) genes, and knocking-in a foreign antigen in a safe harbor on Chr 13 using RCL-BC-2 as the parental line.

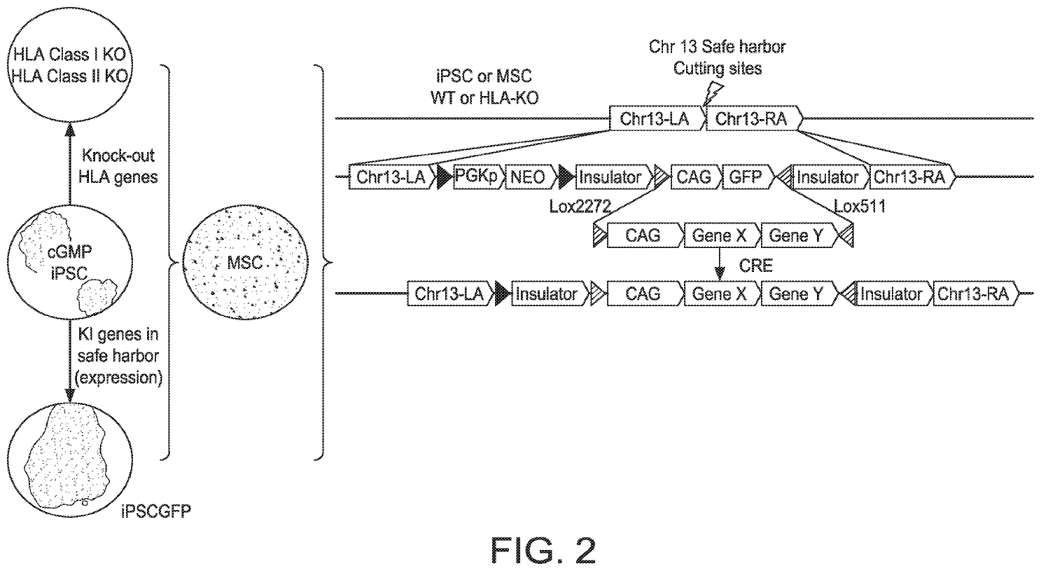

In another nonlimiting embodiment, a floxed construct can be inserted in a safe harbor of a hypoimmunogenic cell to utilize cassette exchange to replace one antigen with another thereby producing multiple cell lines which express multiple epitopes. See FIG. 2. This method allows for rapid production of a population of cells expressing multiple antigens for a polyclonal antibody response. Further, it allows for improved manufacturing as cassette exchange can be performed in cells with limited proliferation potential where clonal selection is difficult. Cassette exchange at the final production step reduces regulatory burden and enables rapid production of novel antigen expressing cells.

The following nonlimiting examples are provided to further illustrate the present invention.

### EXAMPLES

### Example 1: Nucleic Acid Construct for Foreign Antigen Expression of CoV219)

Details of a nonlimiting example of a construct for insertion/integration of a gene for the foreign antigen CoV219 are provided below:

Lox2272-CAG-Cov219 (codon optimized)-SV40pA-
Lox511
(SEQ ID NO: 13)
ataacttcgtataggatactttatacgaagttatatttaaatgacat
tgattattgactagttattaatagtaatcaattacggggtcattagt
tcatagcccatatatggagttccgcgttacataacttacggtaaatg
gcccgcctggctgaccgcccaacgacccccgcccattgacgtcaata
atgacgtatgttcccatagtaacgccaatagggactttccattgacg
tcaatgggtggagtatttacggtaaactgcccacttggcagtacatc
aagtgtatcatatgccaagtacgccccctattgacgtcaatgacggt
aaatggcccgcctggcattatgcccagtacatgaccttatgggactt
tcctacttggcagtacatctacgtattagtcatcgctattaccatgg
tcgaggtgagccccacgttctgcttcactctccccatctcccccccc
tccccacccccaattttgtatttatttattttttaattattttgtgc
agcgatgggggcggggggggggggggcgcgcgccaggcggggcgggg
cggggcgaggggcggggcggggcgaggcggagaggtgcggcggcagc
caatcagagcggcgcgctccgaaagtttctttttatggcgaggcggc
ggcggcggcggccctataaaaagcgaagcgcgcggcgggcgggagtc
gctgcgttgccttcgccccgtgccccgctccgcgccgcctcgcgccg
cccgccccggctctgactgaccgcgttactcccacaggtgagcgggc
gggacggcccttctcctccgggctgtaattagcgcttggtttaatga
cggctcgtttcttttctgtggctgcgtgaaagccttaaagggctccg
ggagggccctttgtgcgggggggagcggctcggggggtgcgtgcgtg
tgtgtgtgcgtggggagcgccgcgtgcggcccgcgctgcccggcggc
tgtgagcgctgcgggcgcggcgcggggctttgtgcgctccgcgtgtg
cgcgaggggagcgcggccgggggcggtgccccgcggtgcgggggggc
tgcgaggggaacaaaggctgcgtgcggggtgtgtgcgtgggggggtg
agcagggggtgtgggcgcggcggtcgggctgtaacccccccctgcac
ccccctccccgagttgctgagcacggcccggcttcgggtgcggggct
ccgtgcggggcgtggcgcggggctcgccgtgccgggcggggggtggc
ggcaggtgggggtgccgggcggggcggggccgcctcgggccggggag
ggctcgggggaggggcgcggcggcccccggagcgccggcggctgtcg
aggcgcaggcgagccgcagccattgccttttatggtaatcgtgcgag
agggcgcagggacttcctttgtcccaaatctgtgcggagccgaaatc
tgggaggcgccgccgcaccccctctagcgggcgcggggcgaagcggt
gcggcgccggcaggaaggaaatgggcggggagggccttcgtgcgtcg
ccgcgccgccgtccccttctccctctccagcctcggggctgtccgcg
gggggacggctgccttcgggggggacggggcagggcggggttcggct
tctggcgtgtgaccggcggctctagagcctctgctaaccatgttcat
gccttcttctttttcctacaggtttacc(CoV219)aaacaaatata
acttcgtataatgtatactatacgaagttat

### Example 2: Production of COVID-19 Antigen iPSC Lines and MSC Derived Therefrom

Several COVID-19 antigen iPSC lines expressing SARS-CoV-2 spike protein, N protein and RNA polymerase were produced. In particular, 3 HLA-KO iPSC lines RCL-BC1-S, RCL-BC1-RNAPol and RCL-BC1-N, expressing SARS-CoV-2 spike protein, N protein and RNA polymerase and 3 control iPSC lines (paternal lines) NCL2-S, NCL2-RNAPol and NCL2-N, expressing SARS-CoV-2 spike protein, N protein and RNA polymerase were produced.

MSCs were then derived from the iPSC lines.

### Example 3: In Vitro Experiments with MSC Derived from HLA-KO iPSC and its Parental iPSC Lines Expressing SARS-CoV-2 Spike Protein (RCL-BC1-S and NCL2-S)

The hypoimmunogenic status and activation of the innate immune response to the MSCs is confirmed.

ELISA-based and PCR-based assays are performed to confirm expression of antigen or antibody.

In vitro challenge assays using sera from immunized mice or expressed antibody using pseudo virus or competitive CFU assays are performed.

### Example 4: In Vivo Experiments with MSC Derived from HLA-KO iPSC and its Parental iPSC Lines Expressing SARS-CoV-2 Spike Protein (RCL-BC1-S and NCL2-S)

The immune response to MSC engineered to express COVID antigens is assessed in mice. Antibody production in mice is tested by ELISA and CFU assays.

Mice (n=5/group) are injected as follows:
Group 1: MSC derived from HLA-null iPSC line stably expressing spike protein;
Group 2: MSC derived from HLA unmodified iPSC stably expressing spike protein;
Group 3: HLA-null MSC transfected with SAM encoding spike protein gene;
Group 4: Unmodified MSC transfected with SAM encoding spike protein gene; and
Group 5: SAM encoding spike protein gene alone.

For these studies, an equal amount of male and female C57BL/6 mice, aged from six to eight weeks, are used. The mice are randomly divided into the above treatment groups. For intramuscular injection, the right hind leg of the mouse is sterilized with 75% alcohol and 1 million modified MSCs are injected through a 1 ml injector. For subcutaneous, a similar number of MSCs are injected into the side abdomen or neck locus.

### Example 5: Additional Antigens

Vaccine tests will be expanded to an influenza vaccine project and determination of immune responses to cells expressing influenza antigen. Mice will be injected similarly to Example 4. Groups are as follows:
HLA-null MSC transfected with SAM encoding hemagglutinin (HA) gene;
Unmodified MSC transfected with SAM encoding HA gene; SAM encoding HA gene alone;

1. A composition comprising a cell modified to be hypoimmunogenic and to express one or more foreign molecules.

2. The composition of claim 1 wherein the foreign molecule is a protein, carbohydrate, nucleotide, cytokines, chemokine, antibody or lectin.

3. The composition of claim 2 wherein the protein is antigenic.

4. The composition of claim 3 wherein the antigenic protein is a viral antigen or an antigen against tumor epitopes.

5. The composition of claim 2 wherein the foreign molecule is an antibody.

6. The composition of claim 1 wherein the cell is modified to be hypoimmunogenic by eliminating or lowering expression of Class I epitopes and Class II epitopes.

7. The composition of claim 1 wherein the cell is a mesenchymal stem cell.

8. A vaccine comprising the composition of claim 1 and a pharmaceutically acceptable carrier.

9. The vaccine of claim 8 further comprising an additional activation agent.

10. The vaccine of claim 9 wherein the activation agent is foreign viral DNA or Poly I:C (Polyinosinic:polycytidylic acid).

11. A method for inducing an immune response against a foreign antigen in a mammal, said method comprising administering to the mammal the composition of claim 1.

12. A method for protecting a mammal from a disease or infection relating to a foreign antigen, said method comprising administering to the mammal the composition of claim 1.

13. A method for producing a vaccine against a foreign antigen, said method comprising modifying a cell to be hypoimmunogenic and to express the foreign antigen.

14. The method of claim 13 wherein the cell is modified to be hypoimmunogenic by eliminating or lowering expression of Class I epitopes and Class II epitopes.

15. A vaccine comprising a cell modified to be hypoimmunogenic and an antigenic peptide or protein.

16. The vaccine of claim 15 wherein the cell is modified to be hypoimmunogenic by eliminating or lowering expression of Class I epitopes and Class II epitopes.

17. A method for enhancing an immune response to an antigenic peptide or protein in a mammal, said method comprising administering to the mammal the vaccine of claim 15.

18. A method for inducing an immune response against a foreign antigen in a mammal, said method comprising administering to the mammal the vaccine of claim 8.

19. A method for protecting a mammal from a disease or infection relating to a foreign antigen, said method comprising administering to the mammal the vaccine of claim 8.


SEQUENCE LISTING (13 sequences)

SEQ ID NO: 1 - RNA, 20 nt, Artificial sequence
  Synthetic
ggccgagaugucucgcuccg

SEQ ID NO: 2 - RNA, 20 nt, Artificial sequence
  Synthetic
acucacgcuggauagccucc

SEQ ID NO: 3 - RNA, 20 nt, Artificial sequence
  Synthetic
cggagcgagagagcacagcg

SEQ ID NO: 4 - DNA, 22 bp, Artificial sequence
  Synthetic
acagcaaactcacccagtctag

SEQ ID NO: 5 - DNA, 21 bp, Artificial sequence
  Synthetic
ccagtctaagggaagcagagc

SEQ ID NO: 6 - DNA, 20 bp, Artificial sequence
  Synthetic
aaactcacccagtctagtgc

SEQ ID NO: 7 - RNA, 20 nt, Artificial sequence
  Synthetic
cacagcugagccccccacug

SEQ ID NO: 8 - RNA, 20 nt, Artificial sequence
  Synthetic
ggcuccugguugaacagcgc

SEQ ID NO: 9 - RNA, 20 nt, Artificial sequence
  Synthetic
ccccuaacauacugggaauc

SEQ ID NO: 10 - DNA, 20 bp, Artificial sequence
  Synthetic
tgagagcttggggtccctta

SEQ ID NO: 11 - DNA, 20 bp, Artificial sequence
  Synthetic
ctgaggcatgttctctgcca

SEQ ID NO: 12 - DNA, 20 bp, Artificial sequence
  Synthetic
ggtaggggcttggagctaac

SEQ ID NO: 13 - DNA, 1761 bp, Artificial sequence
  Synthetic
ataacttcgtataggatactttatacgaagttatatttaaatgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacaggtttaccaaacaaatataacttcgtataatgtatactatacgaagttat